(19) United States
(12) Patent Application Publication
(10) Pub. No.: US 2005/0019272 A1
(43) Pub. Date: Jan. 27, 2005

(54) Portable mouth wash system

(76) Inventor: Johnnie Mae Harrison, Washington, DC (US)

Correspondence Address: RABIN & Berdo, PC, 1101 14TH STREET, NW, SUITE 500, WASHINGTON, DC, 20005, US

(21) Appl. No.: 10/625,641

(22) Filed: Jul. 24, 2003

(51) Int. Cl. A61K 7/16; A61K 9/00; A61C 5/00

(52) U.S. Cl. 424/47; 424/49; 433/217.1

(57) ABSTRACT

A portable mouth wash system provides small quantities of a mouth cleansing liquid and a mouth rinsing liquid in a convenient package. The system includes a dual chamber container having a cylindrical body portion, a transverse divider dividing the body portion into two separate chambers, and sealable openings located at the ends of the body portion, providing access to each of the chambers. One of the chambers contains a mouth cleansing liquid, preferably an aqueous solution of hydrogen peroxide, and the other chamber contains a mouth rinsing liquid, preferably pure water. A closure is provided for sealing each of the sealable openings to contain the mouth cleansing liquid and the mouth rinsing liquids within their respective chambers.

### BACKGROUND OF THE INVENTION

1. Field of the Invention

The invention relates to personal care products, and in particular to a portable mouth wash system providing small quantities of a mouth cleansing liquid and a mouth rinsing liquid in a convenient dual chamber container.

2. Background Information

An aqueous solution of hydrogen peroxide is often used as an antiseptic mouth wash to promote oral health. Hydrogen peroxide is an oxidizer that can kill the bacteria that causes such problems as bad breath and gum disease. However, hydrogen peroxide is commonly sold in quantities that make it impractical to carry and use while traveling, while on camping trips, and in similar situations.

After using hydrogen peroxide as a mouth wash, it is desirable to wash away the taste by rinsing the mouth with pure (steamed or distilled) water. For travelers, campers and the like, drinking water for this purpose may not be readily available, and like peroxide, drinking water is not usually sold in containers small enough to be conveniently carried on the person.

### SUMMARY OF THE INVENTION

It is, therefore, a principle object of this invention to provide a portable mouth wash system.

It is another object of the invention to provide a portable mouth wash system that solves the above-mentioned problems.

These and other objects of the present invention are accomplished by the portable mouth wash system disclosed herein.

In an exemplary aspect of the invention, a portable mouth wash system comprises: a dual chamber container having a cylindrical body portion, including a first end and a second end being directly opposite to one another, a transverse divider within the body portion dividing the body portion into a first chamber and a second chamber, and a sealable opening in each of the first chamber and the second chamber, the sealable openings being disposed at the first end and the second end of the body portion; a first quantity of mouth cleansing liquid disposed in the first chamber; a second quantity of mouth rinsing liquid disposed in the second chamber; and a closure for sealing each of the sealable openings to contain the mouth cleansing liquid and the mouth rinsing liquids within their respective chambers. In this aspect of the invention, the mouth cleansing liquid is preferably an aqueous solution of hydrogen peroxide and the mouth rinsing liquid is preferably pure water. Further, the dual chamber container is preferably made of a plastic material, and the closure is preferably a pull-tab.

In another exemplary aspect of the invention, a portable mouth wash system comprises: a dual chamber container having a body portion, a divider within the body portion dividing the body portion into a first chamber and a second chamber, and a sealable opening in each of the first chamber and the second chamber; a first quantity of mouth cleansing liquid disposed in the first chamber; a second quantity of mouth rinsing liquid disposed in the second chamber; and a closure for sealing each of the sealable openings to contain the mouth cleansing liquid and the mouth rinsing liquids within their respective chambers. In this aspect of the invention, the mouth cleansing liquid is preferably an aqueous solution of hydrogen peroxide and the mouth rinsing liquid is preferably pure water. Further, the dual chamber container is preferably made of a plastic material, and the closure is preferably a pull-tab. The first chamber may have a volume different from that of the second chamber.

In a further exemplary aspect of the invention, a portable personal care system, comprises: a dual chamber container having a body portion, a divider within the body portion dividing the body portion into a first chamber and a second chamber, and a sealable opening in each of the first chamber and the second chamber; a first personal care liquid disposed in the first chamber; a second personal liquid disposed in the second chamber; and a closure for sealing each of the sealable openings to contain the first and second personal care liquids within their respective chambers. The first and second personal care liquids may be any combination of related personal care products used in liquid form.

### BRIEF DESCRIPTION OF THE DRAWINGS

FIG. 1 is a cross-sectional view of a portable mouth wash system according to the present invention.

### DETAILED DESCRIPTION OF THE PREFERRED EMBODIMENTS

The invention will now be described in more detail by way of example with reference to the embodiment shown in the accompanying figure. It should be kept in mind that the following described embodiment is only presented by way of example and should not be construed as limiting the inventive concept to any particular physical configuration.

Further, if used and unless otherwise stated, the terms “upper,” “lower,” “front,” “back,” “over,” “under,” and similar such terms are not to be construed as limiting the invention to a particular orientation. Instead, these terms are used only on a relative basis.

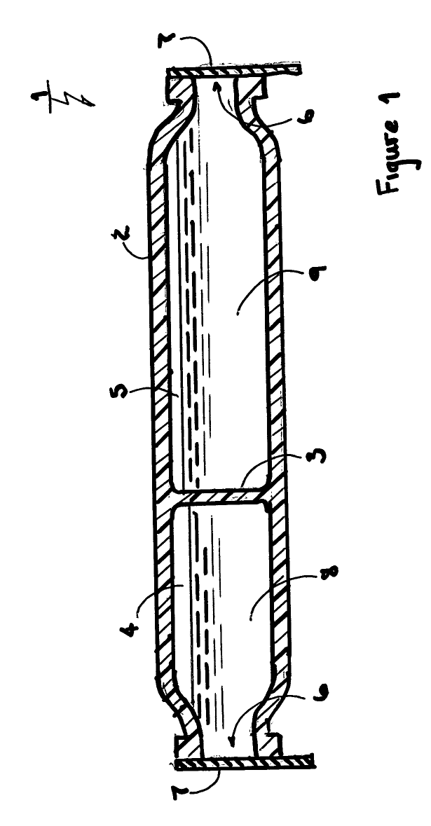

The present invention is directed toward to a portable mouth wash system providing small quantities of a mouth cleansing liquid and a mouth rinsing liquid in a convenient package. With reference to FIG. 1, the portable mouth wash system 1 includes a dual chamber container 2 preferably having a generally cylindrical shape. A transverse divider 3 within the container divides the container into two separate chambers 4 and 5, each of which has a separate sealable opening 6. Preferably, the openings are at the ends of the container. To contain the liquid provided in each chamber, each of the openings has a seal 7.

For the portable mouth wash system, one of the chambers is filled with a mouth cleansing liquid 8, and the other chamber filled with a mouth rinsing liquid 9. Preferably, the mouth cleansing liquid is a 2 percent by volume aqueous solution of hydrogen peroxide, while the mouth rinsing liquid is pure water. The mouth cleansing and the mouth rinsing liquids are preferably non-alcoholic and sugar free. However, the compositions of the mouth cleansing and the mouth rinsing liquids are not limited in any way. Other active ingredients or combinations of active ingredients may be used in the mouth cleansing liquid, including such bacteria-destroying and healing agents as vitamin E, selenium and aloe vera jell, for example. In addition, flavoring and coloring agents, such as spearmint, peppermint, cinnamon and cloves, for example, may be added to the mouth cleansing liquid, the mouth rinsing liquid, or both, to enhance user acceptance. The relative sizes of the chambers can be varied to meet the requirements of the particular mouth cleansing and mouth rinsing liquids being used.

The portable mouth wash system is packaged in a manner that is convenient to carry and use where the usual amenities, such as running water, are not available. The use of a common container with two chambers insures that the two components of the system are always available together. The container is preferably made small enough that it can be easily carried in pocket or purse. The quantities of the two components are preferably chosen so that each system is used for only one treatment and thereafter can be disposed of.

As discussed above, the dual chamber container preferably has a cylindrical shape with the sealable openings for the chambers at opposite ends of the container. However, the inventive concept is not limited to a container of this shape. The container may, for example, consists of two chambers located side-by-side, with the openings at the same end of the package.

For a portable system intended to be carried on the person, the dual chamber container is preferably made of a durable plastic material not prone to breakage or leakage. Dark colored materials are preferable for the storage of hydrogen peroxide, which has some tendency to decompose when exposed to sunlight. A material that can be recycled is especially suitable in applications where the user consumes the contents of the system and disposes of the package after a single use.

The seal used over each opening should be as simple and inexpensive as possible consistent with the application. In the preferred embodiment, which is directed to single-use systems, the seal is a pull-tab fastened by adhesive to the mouth of the sealable opening. The pull-tab may be made of metal foil for strength. The adhesive should be strong enough that the seal will not come loose or leak in the course of normal handling of the package.

For systems intended for multiple uses, a screw cap or other resealable closure is preferably used to seal the openings of the dual chamber container. In other applications, a twist-off or a pop-off cap may be appropriate.

In use, the seal of the first chamber, containing the hydrogen peroxide solution, is removed first and the contents emptied into the mouth. There, it is swished around the teeth and gums for a short period of time, and then spit out (not swallowed). Thereafter, the seal of the second chamber is removed, and the contents used to rinse the mouth, removing the residual hydrogen peroxide. Alternative mouth cleansing and mouth rinsing liquids would be utilized in a similar manner.

It will be recognized by those skilled in the art that the invention in its above-described embodiment is also suitable for use in gargling, at the onset of throat irritation caused by a cold, smoking, etc., and in the treatment of minor cuts and abrasions.

The portable mouth wash system described in this application has a wide variety of practical uses including (but in no way limited to): distribution to military troops in the field; use by workmen on outdoor jobs following meals; during vacationing, camping, picnicking, and at the beach; during travel by air, bus, train, car and boat; use by school children for after lunch dental hygiene; in dental offices as a courtesy to patients; and for distribution in domestic and international health programs.

It should be understood that the invention is not necessarily limited to the specific process, arrangement, materials and components shown and described above, but may be susceptible to numerous variations within the scope of the invention. For example, although the above-described exemplary aspects of the invention are believed to be particularly well suited for a portable mouth wash system, it is contemplated that the concepts of the present invention can be applied in other applications. For example, the concepts of the present application can be utilized whenever it is desired to package together in a convenient portable form any combination of related personal care products used in liquid form.

It will be apparent to one skilled in the art that the manner of making and using the claimed invention has been adequately disclosed in the above-written description of the preferred embodiments taken together with the drawings.

It will be understood that the above description of the preferred embodiments of the present invention are susceptible to various modifications, changes and adaptations, and the same are intended to be comprehended within the meaning and range of equivalents of the appended claims.

1. A portable mouth wash system, comprising:
a dual chamber container having a cylindrical body portion, including a first end and a second end being directly opposite to one another, a transverse divider within the body portion dividing the body portion into a first chamber and a second chamber, and a sealable opening in each of the first chamber and the second chamber, the sealable openings being disposed at the first end and the second end of the body portion;
a first quantity of mouth cleansing liquid disposed in the first chamber;
a second quantity of mouth rinsing liquid disposed in the second chamber; and
a closure for sealing each of the sealable openings to contain the mouth cleansing liquid and the mouth rinsing liquids within their respective chambers.

2. The portable mouth wash system of claim 1, wherein the mouth cleansing liquid is an aqueous solution of hydrogen peroxide.

3. The portable mouth wash system of claim 1, wherein the mouth rinsing liquid is pure water.

4. The portable mouth wash system of claim 1, wherein the dual chamber container is made of a plastic material.

5. The portable mouth wash system of claim 1, wherein the closure is a pull-tab.

6. The portable mouth wash system of claim 1, wherein the closure is a screw cap.

7. A portable mouth wash system, comprising:
a dual chamber container having a body portion, a divider within the body portion dividing the body portion into a first chamber and a second chamber, and a sealable opening in each of the first chamber and the second chamber;
a first quantity of mouth cleansing liquid disposed in the first chamber;
a second quantity of mouth rinsing liquid disposed in the second chamber; and
a closure for sealing each of the sealable openings to contain the mouth cleansing liquid and the mouth rinsing liquids within their respective chambers.

8. The portable mouth wash system of claim 7, wherein the mouth cleansing liquid is an aqueous solution of hydrogen peroxide.

9. The portable mouth wash system of claim 7, wherein the mouth rinsing liquid is pure water.

10. The portable mouth wash system of claim 7, wherein the dual chamber container is made of a plastic material.

11. The portable mouth wash system of claim 7, wherein the closure is a pull-tab.

12. The portable mouth wash system of claim 7, wherein the closure is a screw cap.

13. The portable mouth wash system of claim 7, wherein the first chamber has a first volume and the second chamber has a second volume different from the first volume.

14. A portable personal care system, comprising:
a dual chamber container having a body portion, a divider within the body portion dividing the body portion into a first chamber and a second chamber, and a sealable opening in each of the first chamber and the second chamber;
a first personal care liquid disposed in the first chamber;
a second personal liquid disposed in the second chamber; and
a closure for sealing each of the sealable openings to contain the first and second personal care liquids within their respective chambers.

15. The portable personal care system of claim 14, wherein the body portion is cylindrical in shape and includes a first end and a second end being directly opposite to one another.

16. The portable personal care system of claim 15, wherein the sealable openings are disposed at the first end and the second end of the body portion.

17. The portable personal care system of claim 14, wherein the dual chamber container is made of a plastic material.

18. The portable personal care system of claim 14, wherein the closure is a pull-tab.

19. The portable personal care system of claim 14, wherein the closure is a screw cap.

20. The portable personal care system of claim 14, wherein the first chamber has a first volume and the second chamber has a second volume different from the first volume.
